(19) United States
(12) Patent Application Publication
(10) Pub. No.: US 2023/0045438 A1
(43) Pub. Date: Feb. 9, 2023

(54) SYSTEM AND METHOD FOR PREDICTING LOSS OF FUNCTION CAUSED BY GENETIC VARIANT

(71) Applicant: 3BILLION, Seoul (KR)

(72) Inventors: Kyoungyeul LEE, Seoul (KR); Dong-wook Kim, Seoul (KR)

(73) Assignee: 3BILLION, Seoul (KR)

(21) Appl. No.: 17/817,221

(22) Filed: Aug. 3, 2022

(30) Foreign Application Priority Data
Aug. 4, 2021 (KR) 10-2021-0102589

(51) Int. Cl. G16B 20/00; G16B 40/00; G06F 17/18

(52) U.S. Cl. CPC G16B 20/00; G06F 17/18; G16B 40/00

(57) ABSTRACT

Disclosed herein is a system for predicting a loss of the function of genetic variants. The system includes a loss of function (LoF) prediction unit for calculating a probability that a target genetic variant will cause a loss of function (LoF) in a target gene through logistic regression with respect to a first probability that the target gene will be intolerant of the loss of function and a second probability that the target genetic variant contained in the target gene will be intolerant.

### CROSS-REFERENCE TO RELATED APPLICATIONS

The present application claims the benefit of Korean Patent Application No. 10-2021-0102589, filed Aug. 4, 2021, contents of which are incorporated herein by reference in its entirety.

### BACKGROUND

### 1. Technical Field

The present disclosure relates to a system and a method for predicting a loss of function caused by a genetic variant, and more specifically, to a system and a method for predicting a loss of function caused by a genetic variant, which can calculate a functional loss inducing probability of specific genetic variants through logistic regression.

### 2. Description of Related Art

Due to a genetic testing which has been generalized, a large amount of genome data has been utilized for interpretation of genetic variant so as to interpret influences of genetic variant on a human body very accurately.

In particular, with the development of machine learning technology utilizing a plurality of genome data, an accurate determination of a pathogenic variant has become possible, but there are a number of unclear data points with respect to a specific mechanism causing pathogenicity of a gene.

It is widely known that a variant causing a loss of function (LoF) may cause diseases.

Even in the case that the degree of pathogenicity of the specific genetic variant is digitized through an algorithm or the like, there is lack of grounds for determining a disease inducing variant. However, if an LoF probability can be calculated, a disease inducing mechanism can be specified, thereby enabling a more accurate diagnosis.

Therefore, if the probability that the genetic variant may cause LoF with respect to various genes can be calculated, it can be determined whether or not the genetic variant found in patients with genetic disorders causes the LoF, and it can be utilized for expression and diagnosis of causative genes.

However, it is difficult to estimate the probability of LoF induction of all genetic variants, in that there are very few genetic variants which have turned out to cause the LoF experimentally and clinically, and in that there is little clinical data to allow for estimation as to whether or not a genetic variant causes LoF in a human body.

### SUMMARY

The present disclosure has been made to solve the above-mentioned problems occurring in the prior art, and in an aspect of the present disclosure, it is an object to provide a system and a method for predicting a loss of function caused by a genetic variant, which can be utilized to calculate the probability of a loss of function (LoF) induction of a genetic variant by using a score indicating the degree of pathogenicity of the gene variant and a score indicating the degree of intolerance of genes to the LoF.

To accomplish the above objects, in an aspect of the present disclosure, there is provided a system for predicting a loss of function caused by a genetic variant, the system including: a loss of function (LoF) prediction unit for calculating a probability that a target genetic variant will cause a loss of function (LoF) in a target gene through logistic regression with respect to a first probability that the target gene will be intolerant of the loss of function and a second probability that the target genetic variant contained in the target gene will be intolerant.

In an embodiment of the present invention, the target genetic variant includes a protein truncated variant, in which protein expressed by the variant of a gene is shorter than normal protein.

In an embodiment of the present invention, the first equation is expressed by the following equation:

$${P}_{L o F} = \frac{P \left(i n t o l e r a n t \vert v a r i a n t\right)}{P \left(i n t o l e r a n t \vert L o F\right)} ,$$

wherein ^{P}LoFindicates the probability that the target genetic variant will cause a loss of function (LoF) to the target gene, P(intolerant | LoF)is a first probability, and P(intolerant | variant) is a second probability.

In an embodiment of the present invention, the system further includes: a first characteristic score calculation unit calculating a digitized first characteristic score corresponding to the degree that the target gene is intolerant of the loss of function; and a second characteristic score calculation unit calculating a digitized second characteristic score corresponding to the degree that the target genetic variant has pathogenicity, wherein the first probability is expressed by a × (score_{LoF})^{b}, , the second probability is expressed by c × (score_{pathogenic})^{d}, and score_{Lof} is the first characteristic score, score_{pathogenic} is the second characteristic scores, and a, b, c, and d are respectively predetermined constants.

In an embodiment of the present invention, a log linear model for the first equation includes the following equation:

$$l o g {P}_{L o F} = {β}_{v a r i a n t} \times {X}_{v a r i a n t} + {β}_{g e n e} \times {X}_{g e n e} - l o g Z ,$$

wherein Xᵥₐᵣᵢₐₙₜ is a log value of the second characteristic score, X_{gene} is a log value of the first characteristic score, and βᵥₐᵣᵢₐₙₜ, β_{gene} and Z are respectively predetermined constants.

In an embodiment of the present invention, the first characteristic score includes a score using at least one among a pLI algorithm and an LOEUF algorithm.

In another aspect of the present invention, there is provided a method for predicting a loss of the function of genetic variants, the method including the operation of: calculating a probability that a target genetic variant will cause a loss of function (LoF) in a target gene through logistic regression with respect to a first probability that the target gene will be intolerant of the loss of function and a second probability that the target genetic variant contained in the target gene will be intolerant.

In an embodiment of the present invention, the target genetic variant includes a protein truncated variant, in which protein expressed by the variant of a gene is shorter than normal protein.

In an embodiment of the present invention, the first equation is expressed by the following equation:

$${P}_{L o F} = \frac{P \left(i n t o l e r a n t \vert v a r i a n t\right)}{P \left(i n t o l e r a n t \vert L o F\right)} ,$$

wherein P_{LoF} indicates the probability that the target genetic variant will cause a loss of function (LoF) to the target gene, P(intolerant |LoF) is a first probability, and P(intolerant| variant) is a second probability.

In an embodiment of the present invention, the method further includes the operations of: a first characteristic score calculating operation of calculating a digitized first characteristic score corresponding to the degree that the target gene is intolerant of the loss of function; and a second characteristic score calculating operation of calculating a digitized second characteristic score corresponding to the degree that the target genetic variant has pathogenicity, wherein the first probability is expressed by a × (score _{LoF})^{b}, the second probability is expressed by c × (score _{pathogenic})^{d}, and score_{LoF} is the first characteristic score, score_{pathogenic} is the second characteristic scores, and a, b, c, and d are respectively predetermined constants.

In an embodiment of the present invention, a log linear model for the first equation includes the following equation:

$$l o g {P}_{L o F} = {b}_{v a r i a n t} \times {X}_{v a r i a n t} + {b}_{g e n e} \times {X}_{g e n e} - l o g Z ,$$

wherein, Xᵥₐᵣᵢₐₙₜ is a log value of the second characteristic score, X_{gene} is a log value of the first characteristic score, and βᵥₐᵣᵢₐₙₜ , β_{gene} and Z are respectively predetermined constants.

In an embodiment of the present invention, the first characteristic score includes a score using at least one among a pLI algorithm and an LOEUF algorithm.

### BRIEF DESCRIPTION OF THE DRAWINGS

FIG. 1 is a block diagram illustrating a system for predicting a loss of function caused by a genetic variant according to an embodiment of the present disclosure.

FIG. 2 is a detailed block diagram illustrating a loss of function (LoF) prediction unit of FIG. 1.

FIG. 3 is a flow chart illustrating a method for predicting a loss of function caused by a genetic variant according to an embodiment of the present disclosure.

FIG. 4 is a detailed flow chart illustrating an operation of calculating a probability of causing an LoF in a target gene of FIG. 3.

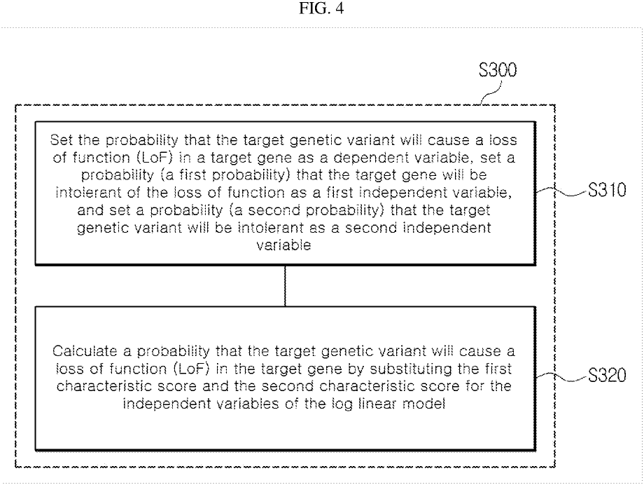

### DETAILED DESCRIPTION

Hereinafter, the present disclosure will be described in more detail with reference to the accompanying drawings. The same reference numerals will be used for the same components in the drawings, and repeated descriptions of the same components will be omitted.

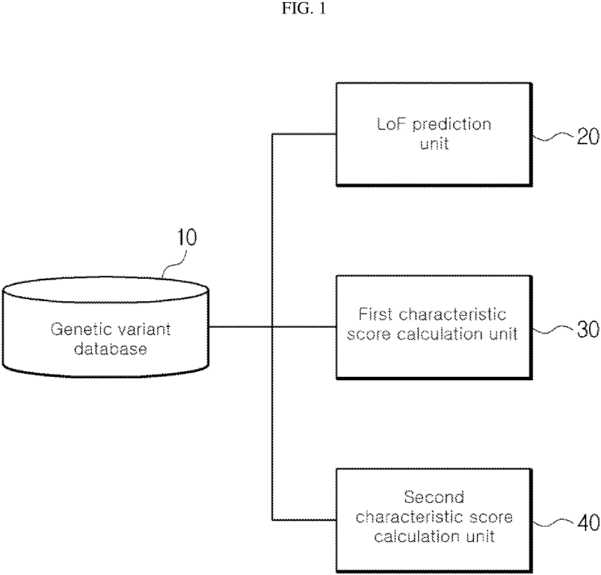

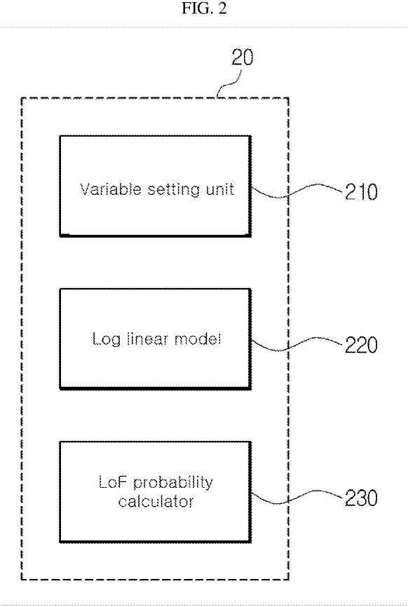

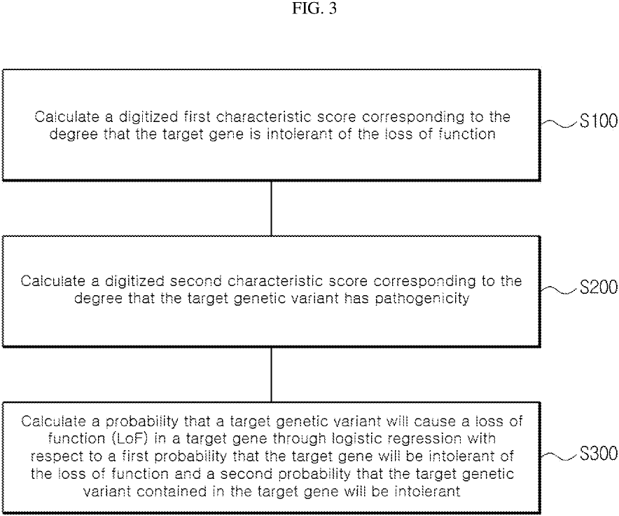

FIG. 1 is a block diagram illustrating a system for predicting a loss of function caused by a genetic variant according to an embodiment of the present disclosure, FIG. 2 is a detailed block diagram illustrating a loss of function (LoF) prediction unit of FIG. 1, and FIG. 3 is a flow chart illustrating a method for predicting a loss of function caused by a genetic variant according to an embodiment of the present disclosure.

Referring to FIGS. 1 and 2, the system for predicting a loss of function caused by a genetic variant according to an embodiment of the present disclosure includes a genetic variant database 10, a loss of function (LoF) prediction unit 20, a first characteristic score calculation unit 30, and a second characteristic score calculation unit 40.

The genetic variant database 10 includes a target genetic variant for allowing the system for predicting a loss of function caused by a genetic variant to calculate a probability that the genetic variant contained in a gene may cause a loss of function (LoF) of the corresponding gene of genetic variant included in a gene, and information on a target gene having the target genetic variant.

DNA contains genetic information of a living thing. A base sequence involved in expression of genetic traits among base sequences of the DNA is referred to as a gene, and a portion that is not involved in the expression of the genetic traits is referred to as a non-coding DNA.

The gene can correspond to a base sequence area over a certain section of the DNA. The gene includes an exon section in which actual genetic information is contained and an intron section that is not involved in expression.

The base sequence or nucleotide sequence refers to a sequence arrangement in which bases as components of nucleotide, which is a base unit of DNA or RNA of nucleic acid, are arranged in order.

A genetic variant or base sequence variant refers to a portion in which there is a difference in sequence between a nucleic acid sequence and a reference sequence, which is a comparison target, and may include substitution, addition or deletion of bases forming a sequence. Such substitution, addition or deletion of bases may be generated by various causes, for instance, structural differences including mutation, cleavage, deletion, duplication, reverse, or translocation of a chromosome.

The term, “loss of function (LoF)” refers to a phenomenon in which a gene loses its original function by a genetic variant.

The LoF prediction unit 20 calculates a probability that a target genetic variant may cause the loss of function in a target gene.

In one embodiment, the LoF prediction unit 20 calculates the probability that a target genetic variant may cause the loss of function in a target gene through logistic regression with respect to a first equation using a first probability and a second probability.

The first probability includes a probability that the target gene is intolerant of the loss of function (LoF).

The second probability includes a probability that the target genetic variant is intolerant.

Here, the LoF intolerance of a gene means that the gene undergoes an influence fatal to survival (it may be extinction or a high probability of disease) when a genetic variant causing a loss of function (LoF) occurs from a specific gene.

In this regard, intolerance gets a higher score when there are less actual cases of genetic variants causing the loss of function (LoF) from genes. The reason is that there is a high probability that the genetic variant may disappear from the natural world by the principle of natural selection in a case in which the genetic variant causing the loss of function (LoF) acts fatally.

As a representative method for calculating a first characteristic score digitized in correspondence to the intolerance degree of a gene with respect to the loss of function (LoF), hereinafter, called the ‘first characteristic score,’ a pLI algorithm may be used. The pLI algorithm is a method to quantify a deviation between the theoretically observable number and the actually observable number of LoF genetic variants in general genomes.

The pLI algorithm is implemented through the following disclosure in the prior art:

Lek, Monkol, et al. “Analysis of protein-coding genetic variant in 60,706 humans.” Nature 536.7616(2016): 285-291. (https://www.nature.com/articles/nature 19057).

In addition, in order to calculate the first characteristic score, an LOEUF algorithm which is similar to the pLI algorithm is used.

The LOEUF algorithm is implemented through the following disclosure in the prior art:

Karczewski, et al. “The mutational constraint spectrum quantified from variant in 141,456 humans.” Nature 581, 434-443 (2020). (https://doi.org/10.1038/s41586-020-2308-7).

Alternatively, in order to calculate the first characteristic score, a method of simply dividing the number of genetic variants causing a loss of function (LoF) actually observed by the number of genetic variants causing a loss of function (LoF) theoretically expected may be used.

Since the first characteristic score is a score defined as a gene unit not as a genetic variant, the extent that each genetic variant causes a loss of function (LoF) cannot be measured.

An intolerance probability of a gene to a loss of function (LoF) can be defined by using the first characteristic score.

On the other hand, since intolerance refers to the extent of having an influence fatal to the survival, the intolerance probability of a genetic variant is strongly associated with a probability that the genetic variant deadly causes a disease.

Therefore, the intolerance probability of the genetic variant (second probability) is proportional to a probability that the genetic variant is pathogenic, that is, a probability that the genetic variant is a pathogenic variant.

In the above conditions, a probability that the target genetic variant will cause a loss of function (LoF) in a target genetic variant can be inversely estimated through the first probability and the second probability.

Specifically, the first equation can be derived through the following procedure.

First, according to all laws of probability, the second probability that the genetic variant is intolerant can be expressed by the following equation:

$$P \left(i n t o l e r a n t \vert v a r i a n t\right) = P \left(i n t o l e r a n t \vert L o F\right) \times P \left(L o F \vert v a r i a n t\right)$$

$$+ P \left(i n t o l e r a n t \left|G o F\right.\right) \times P \left(G o F \left|v a r i a n t\right.\right) .$$

Here, P(intolerant | variant) indicates the probability that the genetic variant is intolerant, that is, the second probability.

P(intolerant | Lof) indicates the probability that the target gene is intolerant of the loss of function (LoF), that is, the first probability.

P(LoF | variant) indicates the probability that the target genetic variant causes the loss of function (LoF) in a target gene, that is, a desired probability.

P(intolerant | GoF) is a probability that the target gene is intolerant of a gain of function (GoF). Here, the gain of function (GoF) is a concept contrary to that of loss of function (LoF), and means that the original function of the gene is further activated by the genetic variant.

P(GoF | variant) indicates a probability that the target genetic variant will cause the gain of function (GoF) in the target gene.

As a first assumption, by the system for predicting the function loss of the genetic variant according to the present disclosure, it is assumed that the target genetic variant for calculating the probability that genetic variant will cause the loss of function to the corresponding gene is limited to a protein truncated variant (PTV).

The PTV means a genetic variant, in which protein expressed by the variant of a gene is shorter than normal protein.

Specifically, the PTV may mean a genetic variant that the length of protein, for instance, the length of an amino acid sequence, expressed from the gene due to at least one among, a frameshift variant, a nonsense variant, a start lost variant, and a splicing variant gets shorter than the length of normal protein.

In a case in which the target genetic variant is limited to the PTV, it is estimated that it is less likely that the PTV will cause the gain of function. Since the probability that the gain of function will be pathogenic is lower than that of the loss of function, it can be assumed that P(intolerant GoF) * P(GoF|variant) approaches 0.

As a second assumption, as mentioned above, the probability (the first probability) that the target gene will be intolerant of the loss of function (LoF) can be expressed as the following equation using the digitized first characteristic score corresponding to the degree that the target gene is intolerant of the function loss:

$$P \left(i n t o l e r a n t \left|L o F\right.\right) = a \times {\left(s c o r {e}_{L o F}\right)}^{b} .$$

Here, score_{LoF} represents the first characteristic score, and a and b represent a predetermined constant.

In addition, since the probability that the target genetic variant will be intolerant (the second probability) may be proportional to the probability that the target genetic variant will be a pathogenic variant, the second probability can be expressed as the following equation using the digitized second characteristic score corresponding to the degree that the target genetic variant has pathogenicity:

$$P \left(i n t o l e r a n t \left|v a r i a n t\right.\right) = c \times {\left(s c o r {e}_{p a t h o g e n i c}\right)}^{d} .$$

Here, score_{pathogenic} represents the second characteristic score, and c and d represent a predetermined constant.

Through the first assumption, the first equation may be represented by the following equation:

$${p}_{L o F} = \frac{p \left(i n t o l e r a n t \left|v a r i a n t\right.\right)}{p \left(i n t o l e r a n t \left|L o F\right.\right)} .$$

Through the second assumption, the first equation may be represented by the following equation:

$${p}_{L o F} = \frac{c \times {\left(s c o r {e}_{p a t h o g e n i c}\right)}^{d}}{a \times {\left(s c o r {e}_{L o F}\right)}^{b}} .$$

Here, P_{LoF} indicates the probability that the target genetic variant will cause a loss of function (LoF) to the target gene.

Next, in order to calculate the probability that the target genetic variant will cause a loss of function (LoF) to the target gene through logistic regression for the first equation, a log linear model 220 for the first equation through log linearization can be expressed as follows:

$$log {P}_{L a F} = {β}_{v a r i a n t} \times {X}_{v a r i a n t} + {β}_{g e n e} \times {X}_{g e n e} - log Z .$$

Here, Xᵥₐᵣᵢₐₙₜ expresses a LOG value of the second characteristic score (i.e., log score _{pathagenic}).

X_{gene} is a LOG value of the first characteristic score (i.e., log score _{LoF}).

βᵥₐᵣᵢₐₙₜ,β_{gene} and Z respectively represent predetermined constants.

For example, βᵥₐᵣᵢₐₙₜ may include the constant D used in the second assumption.

β_{gene} may include the constant -b used in the second assumption.

Z may include the constant a/c used in the second assumption.

Consequently, the probability that the target genetic variant will cause a loss of function (LoF) to the target gene (P(LoF variant) can be calculated through the logistic regression of the digitized first characteristic score corresponding to the degree that the target gene is intolerant of the loss of function (LoF) and the digitized second characteristic score corresponding to the degree that the target genetic variant has pathogenicity.

Under the above theoretical background, the LoF prediction unit 20 includes a variable setting unit 210, a log linear model 220, and a LoF probability calculator 230.

The variable setting unit 210 sets the probability that the target genetic variant will cause a loss of function (LoF) in a target gene as a dependent variable, sets a probability (a first probability) that the target gene will be intolerant of the loss of function as a first independent variable, and sets a probability (a second probability) that the target genetic variant will be intolerant as a second independent variable.

Here, the first probability includes a first characteristic score. In other words, the first probability is represented by using the first characteristic score. The second probability includes a second characteristic score. In other words, the second probability is represented by using the second characteristic score.

The log linear model 220 includes a log linear model modeled through logistic regression analysis with respect to the first equation having a dependent variable, a first independent variable, and a second independent variable.

In one embodiment, the log linear model 220 includes the following equation:

$$log {P}_{L a F} = {β}_{v a r i a n t} \times {X}_{v a r i a n t} + {β}_{g e n e} \times {X}_{g e n e} - log Z .$$

Since the log linear model 220 is the same as described above, a detailed description thereof will be omitted.

The LoF probability calculator 230 can calculate a probability that the target genetic variant will cause a loss of function (LoF) in the target gene by substituting the first characteristic score and the second characteristic score for the independent variables of the log linear model 220.

The first characteristic score calculation unit 30 calculates the digitized first characteristic score corresponding to the degree that the target gene is intolerant of the loss of function.

In one embodiment, the first characteristic score is calculated by using an in-silico tool using a computer simulation. The calculation of the first characteristic score by using the in-silico tool may use an algorithm to quantify a deviation between the theoretically observable number and the actually observable number of LoF genetic variants in general genomes.

For instance, the algorithm used for calculation of the first characteristic score includes at least one algorithm among pLI and LOEUF.

The second characteristic score calculation unit 40 calculates the digitized second characteristic score corresponding to the degree that the target genetic variant has a pathogenicity.

In one embodiment, the second characteristic score is calculated by using an in-silico tool using a computer simulation. The calculation of the second characteristic score by using the in-silico tool may use an algorithm to digitize pathogenicity of variation.

For example, the algorithm used for calculating the characteristic score of the variants may include at least one algorithm among REVEL, SIFT, PrimateAI, DANN, PolyPhen, PolyPhen-2, 3CNET, MAPP, Logre, Mutation Assessor, Condel, GERP, CADD, MutationTaster, MutationTaster2, PROVEAN, PMuit, SNPeffect, fathmm, MSRV, Align-GVGD, Eigen, LRT, MetaLR, MetaSVM, MutPred, PANTHER, Parepro, phastCons, PhD--SNP, phyloP, PON-P, PON-P2, SiPhy, SNAP, SNPs&GO, VEST4, SNAP2, CAROL, PaPI, SInBaD, VAAST, CHASM, mCluster, nsSNPAnayzer, SAAPpred, HanSa, CanPredict, FIS and BONGO.

The algorithm for digitizing the pathogenicity of the genetic variant, which is applied to the present disclosure, can be implemented by the following known prior art documents, and the detailed description related thereto can be omitted.

REVEL (Ioannidis, Nilah M., et al. REVEL: an Ensemble Method for Predicting the Pathogenicity of Rare Missense Variants.__ AGHG 2016, https://sites.goog!e.com/site/revelgenomics/), SIFT (Sorting Intolerant From Tolerant, Pauline C et al., Genome Res. 2001 May; 11(5): 863-874; Pauline C et al., Genome Res. 2002 March; 12(3): 436-446; Jing Hulet al., Genome Biol. 2012; 13(2): R9), PrimateAI(Illumina Company’s deep learning model for pathogenicity prediction) DANN (Quang, Daniel, Yifei Chen, and Xiaohui Xie. DANN: a deep learning approach for annotating the pathogenicity of genetic variants. Bioinformatics 2014: btu703., https://cbcl.ics.uci.edu/public_data/DANN/), PolyPhen, PolyPhen-2 (Polymorphism Phenotyping, Ramensky V et al., Nucleic Acids Res. 2002 September 1; 30(17): 3894-3900; Adzhubei IA et al., Nat Methods 7(4):248-249 (2010)), 3Cnet(3Cnet: Pathogenicity prediction of human variants using knowledge transfer with deep recurrent neural networks, Dhong-gun Won, Kyoungyeul Lee, bioRxiv 2020.09.27.302927; doi: https://doi.org/10.1101/2020.09.27.302927), MAPP (Eric A. et al., Multivariate Analysis of Protein Polymorphism, Genome Res. 2005; 1 5:978-986), Logre (Log R Pfam E-value, Clifford R.J et al., Bioinformatics 2004;20:1006-1014), Mutation Assessor (Reva B et al., Genome Biol. 2007;8:R232, http://mutationassessor.org/), Condel (Gonzalez-Perez A et al.,The American Journal of Human Genetics 2011;88:440.-449, http://bg.upf.edu/fannsdb/), GERP (Cooper et al., Genomic Evolutionary Rate Profiling, Genome Res. 2005;15:901-913, http://mendel.stanford.edu/SidowLab/downloads/gerp/), CADD (Combined Annotation-Dependent Depletion, http://cadd.gs.washington.edu/), MutationTaster, MutationTaster2 (Schwarz et al., MutationTaster2: mutation prediction for the deep-sequencing age. Nature Methods 2014;11:361-362, http://www.mutationtaster.org/), PROVEAN (Choi et al., PLoS One. 2012;7(10):e46688), PMuit (Ferrer-Costa et al., Proteins 2004;57(4):811-819, http://mmb.pcb.ub.es/PMut/), SNPeffect (Reumers et al., Bioinformatics. 2006;22(17):2183-2185, http://snpeffect.vib.be), fathmm (Shihab et al., Functional Analysis through Hidden Markov Models, Hum Mutat 2013;34:57-65, http://fathmm.biocompute.org.uk/), MSRV (Jiang, R. et al. Sequence-based prioritization of nonsynonymous single-nucleotide polymorphisms for the study of disease mutations. Am J Hum Genet 2007;81:346-360, http://msms.usc.edu/msrv/), Align-GVGD (Tavtigian, Sean V., et al. Comprehensive statistical study of 452 BRCA1 missense substitutions with classification of eight recurrent substitutions as neutral. Journal of medical genetics 2006:295-305., http://agvgd.hci.utah.edu/), Eigen (lonita-Laza, Iuliana, et al. A spectral approach integrating functional genomic annotations for coding and noncoding variants. Nature genetics (2016):214-220., http://www.columbia.edu/~ii2135/eigen.html), LRT (Chun, Sung, and Justin C. Fay. Identification of deleterious mutations within three human genomes. Genome Res. 2009: 1553-1561., http://www.genetics.wustl.edu/jflab/lrt__.query.html), MetaLR (Dong, Chengliang, et al. Comparison and integration of deleteriousness prediction methods for nonsynonymous SNVs in whole exome sequencing studies. Human molecular genetics 2015;24(8):2125-2137), MetaSVM (Dong, Chengliang, et al. Comparison and integration of deleteriousness prediction methods for nonsynonymous SNVs in whole exome sequencing studies. Human molecular genetics 2015;24(8):2125-2137), MutPred (Mort, Matthew, et al. MutPred Splice: machine learning-based prediction of exonic variants that disrupt splicing. Genome Biology 2014-(15)1-.1http://www.mutdb.org/mutpredsplice/about.htm),PANTHER (Mi, Huaiyu, et al. The PANTHER database of protein families, subfamilies, functions and pathways. Nucleic Acids Research 2005;(33)suppl I.-D284-D288., http://www.pantherdb.org/tools/csnpScoreForm.jsp), Parepro (Tian, Jian, et al. Predicting the phenotypic effects of non-synonymous single nucleotide polymorphisms based on support vector machines. BMC bioinformatics 2007; 8.1, http://www.mobioinfor.cn_{/}parepro/contact.htm), phastCons (Siepel, Adam, et al. Evolutionarily conserved elements in vertebrate, insect, worm, and yeast genomes. Genome Res. 2005;915)8:1034-1050, http://compgen.csh1.edu/phast/), PhD-SNP (Capriotti, E., Calabrese, R., Casadio, R. Predicting the insurgence of human genetic diseases associated to single point protein mutations with support vector machines and evolutionary information. Bioinformatics 2006;22:2729-2734., http://stips.biofold.org/plidsnpl/), phyloP (Pollard, Katherine S., et al. Detection of nonneutral substitution rates on mammalian phylogenies. Genome Res. 2010;(20)1:110-121., http://compgen.cshl.edu/phast/background.php), PON-P (Niroula, Abhishek, Siddhaling Urolagin, and Mauno Vihinen. PON--P2: prediction method for fast and reliable identification of harmful variants. PLoS One 2015;(10)2:e0117380., http://structure.bmc.1u.se/PON.-P2/), SiPhy (Garber, Manuel, et al. Identifying novel constrained elements by exploiting biased substitution patterns. Bioinformatics 2009;(25)12:i54-i62, http://portals.broadinstitute.org/genome_bio/siphy/documentation.html), SNAP (Bromberg,Y. and Rost,B. SNAP: predict effect of non-synonymous polymorphisms on function. Nucleic Acids Res. 2007;35:3823-3835,w http://www.rostlab.org/services/SNAP), SNPs&GO (Remo Calabrese, Emidio Capriotti, Piero Fariselli, Pier Luigi Martelli, and Rita Casadio. Functional annotations improve the predictive score of human disease-related mutations in proteins. Human Mutatation 2009;30:1237- 1244, http://snps.biofold.org/snps-and-go/), VEST4 (Carter H, Douville C. Stenson P, Cooper D, Karchin R Identifying Mendelian disease genes with the Variant Effect Scoring Tool BMC Genomics 2013;14(Suppl 3):S3), SNAP2 (Yana Bromberg, Guy Yachdav, and Burkhard Rost. SNAP predicts effect of mutations on protein function. Bioinformatics 2008;24:2397-2398, http://www.rostlab.org/services/SNAP), CAROL (Lopes MC, Joyce C, Ritchie GR, John SL, Cunningham F et al. A combined functional annotation score for non-synonymous variants, http://www.sanger.ac.uk/science/tools/carol), PaPI (Limongelli, Ivan, Simone Marini, and Riccardo Bellazzi. PaPI: pseudo amino acid composition to score human protein-coding variants. BMC bioinformatics 2015;(16)1:1, http://papi.unipv.it/), SInBaD (Lehmann, Kjong- Van, and Ting Chen. Exploring functional variant discovery in non-coding regions with SInBaD. Nucleic Acids Research 2013;(41)1 :e7-e7, http://tingchenlab.cmb.usc.edu/sinbad/), VAAST (Hu, Hao, et al. VAAST 2.0: Improved variant classification and disease..]gene identification using a conservation_]controlled amino acid substitution matrix. Genetic epidemiology 2013;(37)6:622-634, http)://www.yandell--lab.org/software/vaast.html), CHASM (Carter H, Chen S, Isik L, Tyekucheva S, Velculescu VE, Kinzler KW, Vogelstein B, Karchin R Cancer-specific high-throughput annotation of somatic mutations: computational prediction of driver missense mutations Cancer Res 2009;69(16):6660-7, http://www.cravat.us), mCluster (Yue P, Forrest WF, Kaminker JS, Lohr S, Zhang Z, Cavet G: Inferring the functional effects of mutation through clusters of mutations in homologous proteins. Human mutation. 2010;31(3):264-271. 10.1002/humu.21194.), nsSNPAnayzer (Lei Bao, Mi Zhou, and Yan Cui nsSNPAnalyzer: identifying disease-associated nonsynonymous single nucleotide polymorphisms. Nucleic Acids Res 2005;33:480-482, http://snpanalyzer.uthsc.edu/), SAAPpred (Nouf S Al-Numair and Andrew C R Martin. The SAAP pipeline and database: tools to analyze the impact and predict the pathogenicity of mutations. BMC Genomics 2013; 14(3):1-11, www.bioinf.org.uk/saap/dap/), HanSa (Acharya V. and Nagarajaram H.A. Hansa An automated method for discriminating disease and neutral human nsSNPs. Human Mutation 2012;2:332-337, hansa.cdfd.org.in:8080/), CanPredict (Kaminker,J.S. et al. CanPredict: a computational tool for predicting cancer-associated missense mutations. Nucleic Acids Res., 2007;35:595:598, http://pgws.nci.nih.gov/cgi-bin/GeneViewer.cgi_), FIS (Boris Reva, Yevgeniy Antipin, and Chris Sander. Predicting the functional impact of protein mutations: Application to cancer genomics. Nucleic Acids Res 2011 ;39:e118-e118.), BONGO (Cheng T.M.K., Lu Y-E, Vendruscolo M., Lio P., Blundell T.L. Prediction by graph theoretic measures of structural effects in proteins arising from non-synonymous single nucleotide polymorphisms. PLoS Comp Biology 2008;(4)7:e1000135, http://www.bongo.cl.cam.ac.uk/Bongo2/Bongo .htm)

While the exemplary embodiments of the present disclosure have been described in more detail with reference to the accompanying drawings, it will be understood by those of ordinary skill in the art that various modifications, changes and equivalents may be made without deviating from the spirit or scope of the disclosure described in the following claims.

### ADVANTAGEOUS EFFECTS

As described above, the system and the method for predicting a loss of the function of genetic variants according to the present disclosure can specify the probability of a disease induction mechanism by calculating the LoF induction probability of genetic variants, thereby improving accuracy in diagnosis.

In addition, the system and the method for predicting a loss of the function of genetic variants according to the present disclosure can specify an area serving an important role for genetic functions through LoF scores varying according to the position of the genetic variant, thereby providing important information for development of new medicine targeting protein.

1. A system for predicting a loss of the function of genetic variants, the system comprising:
a loss of function (LoF) prediction unit for calculating a probability that a target genetic variant will cause a loss of function (LoF) in a target gene through logistic regression with respect to a first probability that the target gene will be intolerant of the loss of function and a second probability that the target genetic variant contained in the target gene will be intolerant.

2. The system according to claim 1, wherein the target genetic variant includes a protein truncated variant, in which protein expressed by the variant of a gene is shorter than normal protein.

3. The system according to claim 2, wherein the first equation is expressed by the following equation:
$${P}_{L o F} = \frac{P \left(i n t o l e r a n t \vert v a r i a n t\right)}{P \left(i n t o l e r a n t \vert L o F\right)} ,$$
wherein P_{LoF} indicates the probability that the target genetic variant will cause a loss of function (LoF) to the target gene, P(intolerant | LoF) is a first probability, and P(intolerant | variant) is a second probability.

4. The system according to claim 3, further comprising:
a first characteristic score calculation unit calculating a digitized first characteristic score corresponding to the degree that the target gene is intolerant of the loss of function; and
a second characteristic score calculation unit calculating a digitized second characteristic score corresponding to the degree that the target genetic variant has pathogenicity,
wherein the first probability is expressed by a × (score_{LoF})^{b},
wherein the second probability is expressed by c × (score_{pathogenic})^{d},and
wherein score_{LoF} is the first characteristic score, score_{pathogenic} is the second characteristic scores, and a, b, c, and d are respectively predetermined constants.

5. The system according to claim 4, wherein a log linear model for the first equation includes the following equation:
$$l o g {P}_{L a F} = {β}_{v a r i a n t} \times {X}_{v a r i a n t} + {β}_{g e n e} \times {X}_{g e n e} - l o g Z$$
, and
wherein, Xᵥₐᵣᵢₐₙₜ is a log value of the second characteristic score, X_{gene} is a log value of the first characteristic score, and βᵥₐᵣᵢₐₙₜ,β_{gene} and Z are respectively predetermined constants.

6. The system according to claim 4, wherein the first characteristic score includes a score using at least one among a pLI algorithm and an LOEUF algorithm.

7. A method for predicting a loss of the function of genetic variants, the method comprising the operation of:
calculating a probability that a target genetic variant will cause a loss of function (LoF) in a target gene through logistic regression with respect to a first probability that the target gene will be intolerant of the loss of function and a second probability that the target genetic variant contained in the target gene will be intolerant.

8. The method according to claim 7, wherein the target genetic variant includes a protein truncated variant, in which protein expressed by the variant of a gene is shorter than normal protein.

9. The system according to claim 8, wherein the first equation is expressed by the following equation:
$${P}_{L o F} = \frac{P \left(i n t o l e r a n t \vert v a r i a n t\right)}{P \left(i n t o l e r a n t \vert L o F\right)} ,$$
wherein P_{LoF}indicates the probability that the target genetic variant will cause a loss of function (LoF) to the target gene, P(intolerant | LoF) is a first probability, and P(intolerant | variant) is a second probability.

10. The method according to claim 9, further comprising the operations of:
a first characteristic score calculating operation of calculating a digitized first characteristic score corresponding to the degree that the target gene is intolerant of the loss of function; and
a second characteristic score calculating operation of calculating a digitized second characteristic score corresponding to the degree that the target genetic variant has pathogenicity,
wherein the first probability is expressed by a × (score_{LoF})^{b}
wherein the second probability is expressed by c × (score_{pathogenic})^{d}, and
wherein score_{LoF}is the first characteristic score, score_{pathogenic} is the second characteristic scores, and a, b, c, and d are respectively predetermined constants.

11. The method according to claim 10, wherein a log linear model for the first equation includes the following equation:
$$log {P}_{L a F} = {β}_{v a r i a n t} \times {X}_{v a r i a n t} + {β}_{g e n e} \times {X}_{g e n e} - log Z$$
, and wherein, Xᵥₐᵣᵢₐₙₜ is a log value of the second characteristic score, X_{gene}is a log value of the first characteristic score, and βᵥₐᵣᵢₐₙₜ, β_{gene} and Z are respectively predetermined constants.

12. The method according to claim 10, wherein the first characteristic score includes a score using at least one among a pLI algorithm and an LOEUF algorithm.
